(19) United States
(12) Patent Application Publication
(10) Pub. No.: US 2024/0407660 A1
(43) Pub. Date: Dec. 12, 2024

(54) METHOD AND SYSTEM FOR PERFORMING IMAGING USING LOW-FREQUENCY ELECTROMAGNETIC FIELDS

(71) Applicant: ESI Imaging Inc., Waterloo (CA)

(72) Inventors: Omar Mustafa RAMAHI, Waterloo (CA); Dawood ALSAEDI, Makkah (SA); Hamid Akbari CHELARESI, Waterloo (CA)

(21) Appl. No.: 18/740,738

(22) Filed: Jun. 12, 2024

Related U.S. Application Data
(60) Provisional application No. 63/577,998, filed on Jun. 12, 2023.

(51) Int. Cl. A61B 5/05; A61B 5/00

(52) U.S. Cl. CPC A61B 5/05; A61B 5/004

(57) ABSTRACT

The disclosure is directed at a system and method for imaging an object of interest. The system includes a low-frequency radiation source for transmitting a set of low-frequency electromagnetic waves towards the object of interest wherein a size of each of the set of low-frequency electromagnetic waves are larger than a volume of the object of interest; a metasurface for receiving the set of low-frequency electromagnetic waves after they have passed through or around the object of interest to generate impression signals, the metasurface including a set of unitcells; and a processor for generating at least one impression of the object of interest based on the impression signals.

### CROSS-REFERENCE TO OTHER APPLICATIONS

The disclosure claims priority from US Provisional Application No. 63,577,998 filed Jun. 12, 2023 which is hereby incorporated by reference.

### FIELD

The disclosure is generally directed at imaging technologies and, more specifically, at a method and system for performing imaging using low-frequency electromagnetic fields.

### BACKGROUND

In the field of imaging, there are many different solutions that are used, however, depending on the application, these solutions suffer from different problems. For instance, in the medical field, there is a desire to reduce the harmful exposure of patients to X-ray imaging due to the negative impact of X-rays. This may be complicated by the fact that somewhat regular imaging may be required or beneficial for the early detection of cancer in patients.

After skin cancer, breast cancer is the most common cancer diagnosed in women and is the cause of a poor quality of life for many women. Current technologies have been facing challenges that are associated with at least one of health-related issues, scanning time, and affordability.

One current method for cancer or tumor detection is X-ray mammography. Although this technology is suitable for detecting malignant tissues in low-density breasts, it has a potentially harmful effect on the patient due to ionizing radiation. For high-density breasts, diagnosis of cancerous tumors is challenging using this technology due to the high overlap between fat and malignant tissues. Magnetic resonance imaging (MRI) is also used as a complementary diagnosing tool, providing higher accuracy than X-ray mammography, especially for high-density breasts, however, using MRI technology is costly and therefore expensive when used for regular screening, particularly in low-income communities. Ultrasound is also used for breast-cancer diagnoses; however, its accuracy in detecting tumors depends on the radiologist's expertise.

In recent years, breast cancer detection techniques based on microwave imaging (MWI) have been introduced as an alternative method of testing or detection. MWI uses non-ionizing electromagnetic (EM) waves instead of potentially hazardous ionizing waves. In addition, low-frequency electromagnetic excitation allows for deeper penetration, thereby increasing the ability to diagnose anomalies buried deep inside denser breasts. Furthermore, MWI takes advantage of low-cost system integration.

Therefore, there is provided a novel method and system for performing imaging using low-frequency electromagnetic fields that overcomes disadvantages of current systems.

### SUMMARY

The disclosure is directed at a novel method and system for performing imaging using low-frequency electromagnetic fields.

In one aspect of the disclosure, there is provided a system for imaging an object of interest including a low-frequency radiation source for transmitting a set of low-frequency electromagnetic waves towards the object of interest wherein a size of each of the set of low-frequency electromagnetic waves are larger than a volume of the object of interest; a metasurface for receiving the set of low-frequency electromagnetic waves after they have passed through or around the object of interest to generate impression signals, the metasurface including a set of unitcells; and a processor for generating at least one impression of the object of interest based on the impression signals.

In another aspect, the disclosure further includes a signal generator for providing an input to the low-frequency radiation source. In yet another aspect, the input is a continuous wave signal. In a further aspect, the input is instructions to generate the set of low-frequency electromagnetic waves.

In yet a further aspect, the disclosure includes a display for displaying the at least one impression of the object of interest. In yet another aspect, each of the set of unitcells is smaller than the size of each of the low-frequency electromagnetic waves. In yet a further aspect, the set of unitcells are arranged in an array. In another aspect, each of the set of unitcells includes a reactive portion; and a non-reactive portion. In a further aspect, the reactive portion includes an outer portion; and an inner portion. In another aspect, the reactive portion further includes a set of vias connected to the outer portion.

In aspect, each of the set of unitcells includes surface mounted components.

In another aspect of the disclosure, there is provided a method of imaging an object of interest including transmitting low-frequency electromagnetic waves at an object of interest wherein a size of each of the set of low-frequency electromagnetic waves are larger than a volume of the object of interest; capturing the low-frequency electromagnetic waves that pass through or around the object of interest with a metasurface including a set of unitcells; generating impression signals based on the captured low-frequency electromagnetic waves; and generating an impression based on the impression signals.

In another aspect, each of the set of unitcells is smaller than the size of each of the low-frequency electromagnetic waves. In a further aspect, generating impression signals includes processing the captured low-frequency electromagnetic waves. In yet another aspect, transmitting low-frequency electromagnetic waves includes receiving an input at a low-frequency radiation source; generating the low-frequency electromagnetic waves based on the input; and transmitting the low-frequency electromagnetic waves at the object of interest.

### DESCRIPTION OF THE DRAWINGS

Some embodiments of the present disclosure are illustrated as an example and are not limited by the figures of the accompanying drawings, in which like references may indicate similar elements and in which:

FIG. 1a is a schematic diagram of a system for imaging using low-frequency electromagnetic waves;

FIG. 1b is a schematic diagram of a system for mammography using low-frequency electromagnetic fields;

FIG. 1c is a schematic diagram of another embodiment of a system for imaging a pipe using low-frequency electromagnetic waves;

FIG. 2a is a perspective view of a metasurface of use in the system of FIG. 1a;

FIGS. 2b to 2d are different views of a unitcell;

FIG. 3 is a table showing parameters for one embodiment of a unitcell;

FIG. 4a is a chart showing |S11| measurements for different values of thickness;

FIG. 4b is a chart showing |S11| measurements for different values of L;

FIG. 4c is a chart showing |S11| measurements where R1=R2;

FIG. 4d is a chart showing values for |S11| and power for a unitcell;

FIG. 5a is a top view of a metasurface;

FIG. 5b is a bottom view of a metasurface;

FIG. 5c is a schematic diagram showing a scanning technique using the metasurface;

FIG. 5d is a sematic diagram of a breast model;

FIG. 6a is a simulation model of a healthy breast;

FIG. 6b is a simulation model of a tumorous breast;

FIG. 6c is an impression of the healthy breast of FIG. 6a using the system of the disclosure;

FIG. 6d is an impression of the tumourous breast of FIG. 6b using the system of the disclosure;

FIG. 6e is a chart showing where test tumours are located within a breast model;

FIGS. 7a to 7d are breast models showing a 10 mm tumour in different locations;

FIGS. 8a to 8d are breast models showing a 7.5 mm tumour in different locations;

FIGS. 9a to 9d are breast models showing a 5 mm tumour in different locations;

FIGS. 9e to 9h are impressions of the breast models of FIGS. 9a to 9d;

FIG. 10a is a perspective view of a simulation setup;

FIG. 10b is an impression received from the simulation setup of FIG. 10a;

FIG. 11 is a pair of schematic diagrams of breast models; and

FIG. 12 is a flowchart outlining a method of imaging a body part for at least one anomaly.

### DETAILED DESCRIPTION OF THE EMBODIMENTS

The disclosure is directed at a method and system for performing imaging of an object of interest using low-frequency electromagnetic waves or fields. In one embodiment, the disclosure includes a transmitter that transmits and/or directs low-frequency electromagnetic waves towards the object of interest and a metasurface that receives or senses the electromagnetic waves after they have passed through and/or passed by the object of interest. The electromagnetic waves are larger than a volume or size of the object of interest. In other embodiments, a size of unitcells within the metasurface are smaller than the size of the electromagnetic waves. In some embodiments, the low-frequency electromagnetic waves are between about 100 MHz to about 200 MHz.

In some embodiments, the disclosure is used to detect anomalies in a body part of interest using a low-frequency electromagnetic field for example, as a mammography apparatus, and a method for using the mammography apparatus. In some embodiments, the disclosure can be seen as an apparatus for early breast cancer screening or tumor detection.

When the disclosure is used as a mammography apparatus, the disclosure obtains an impression of a body part, such as a breast, that correlates to the constituents of the body part using the system and method of the disclosure. One advantage of the disclosure is that it reduces or eliminates the exposure of a patient to harmful radiation. Another advantage is that the patient may have more frequent exams due to the reliance of the disclosure on low-frequency microwaves and not harmful higher frequency radiation.

In other embodiments, the disclosure may be used to detect cracks or fractures in other body parts of interest. In yet further embodiments, the disclosure may be used to detect cracks or fractures in pipes, walls and the like. In yet other embodiments, the disclosure may be used to examine a purity of rubber or plastic materials.

In an embodiment, the disclosure captures an impression or image of an area or item, such as, but not limited to, a body part or a set of pipes, using a metasurface including a set of unitcells or electrically-small resonators stitched together to provide a surface that has a high or strong electromagnetic energy absorbance. One advantage of the disclosure, when used for imaging a breast, is the provision of a safe and inexpensive method and system for early detection or screening of anomalies in a body part, such as, but not limited to, a tumor in a patient's breast which may lead to breast cancer.

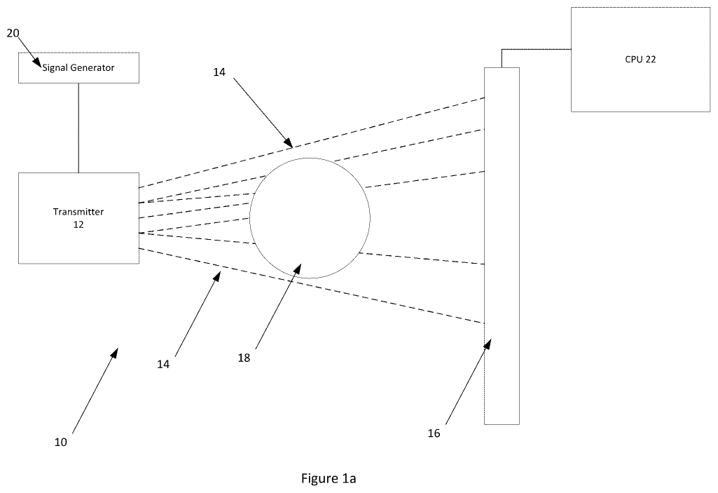

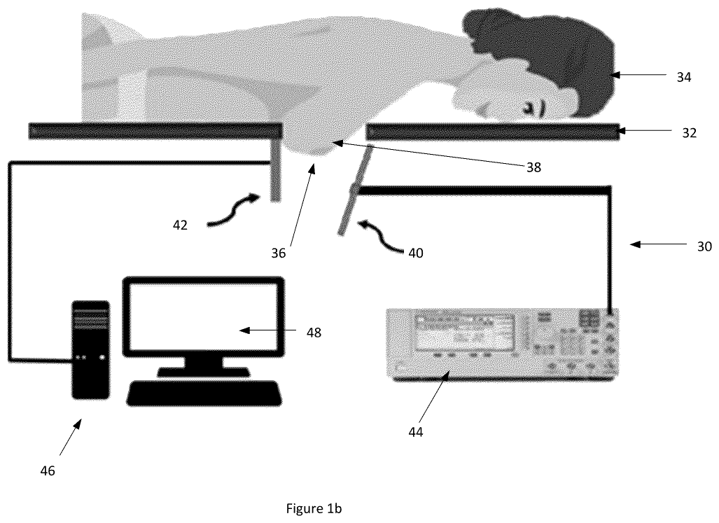

Turning to FIG. 1a, a schematic diagram showing a first embodiment of a system for imaging using low-frequency electromagnetic waves in accordance with the disclosure is shown. The system 10 includes a transmitter 12 or transmitting component that transmits low frequency electromagnetic waves 14 and at least one metasurface 16 that receives low frequency waves that are transmitted by the transmitter 12 after they pass by or through an object of interest 18. In embodiments, a size of the electromagnetic waves are larger than the volume of the object of interest.

The transmitter 12 is connected to a signal generating component 20 that provides a signal or instructions to the transmitter 12. The signal generating component 18 may receive instructions with respect to a frequency level or a shape of the low-frequency waves that are being transmitted by the transmitter 12. The metasurface 16 is connected to a central processing unit (CPU) 22 which processes the signals received by the metasurface 16. As can be seen, in the current embodiment, the metasurface 16 is located on an opposite side of the object of interest 18 from the transmitter 12. The metasurface 16 includes a set of unitcells (as described below) where each of the unitcells is smaller than size of the electromagnetic waves.

As shown in FIG. 1a, in operation, the transmitter 12 transmits the low-frequency waves at the object of interest 18. Depending on the characteristics of the object of interest 18, such as its transparency or opaqueness, a portion of the low-frequency electromagnetic waves may pass through the object of interest 18 while, in other test or operational environments, the low-frequency electromagnetic waves 14 may be blocked by the object of interest 18. Other low-frequency waves 14 may simply pass by the object of interest 18. The sensed low-frequency electromagnetic waves, which may be seen as a set of received or impression signals are then passed to the CPU 22 which processes the received signals to generate an image or impression of the object of interest 18. In other embodiments, the set of received signals may be processed before they are transmitted to the CPU 22.

Turning to FIG. 1b, a side view of another embodiment of an imaging system using low-frequency electromagnetic waves is shown. The current embodiment may be seen as a system for imaging a body part using low-frequency electromagnetic waves or a mammography apparatus using low-frequency electromagnetic waves. In some embodiments, the images generated by the system may be used to detect anomalies in the body part. The environment in which the imaging system 30 can be used includes a platform 32 on which a patient 34 can be positioned. The platform 32 includes an opening 36 for receiving a body part 38, or item of interest, that is being examined or imaged by the apparatus or system 30. In this example, the body part is the breast of the patient 34.

On one side of the opening 36, the system 30 includes a low-frequency transmitter, or radiation source 40, such as, but not limited to, an electrically small (ES) dipole antenna operating as a near-field source. On an opposite side of the opening 36, the system 30 includes at least one metasurface 42. In operation, the radiation source 40 transmits low-frequency electromagnetic waves towards the body part 38 and the at least one metasurface 42.

In the current embodiment, as ES antennas are fundamentally low-efficient whereby a few percentage of pumped power from a signal generator will be radiated outside, the radiated power (transmitted by the transmitter 40) can be increased by selecting a desired ES antenna or by using a high power signal as the excitation source. In the current embodiment, the radiation source 40 may include a power amplifier in series with an isolator that are connected to the ES antenna. The radiated low-frequency electromagnetic waves are directed towards the body part 38 and penetrate into the body part, such as a female breast. In some embodiments, the low-frequency radiation source 40 may be a horn antenna that is used to illuminate the body part 38 with low-energy microwave radiation over a narrow frequency band.

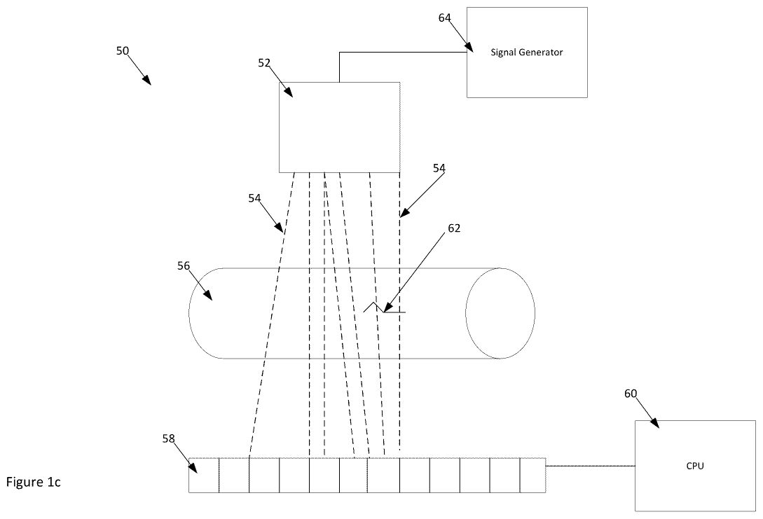

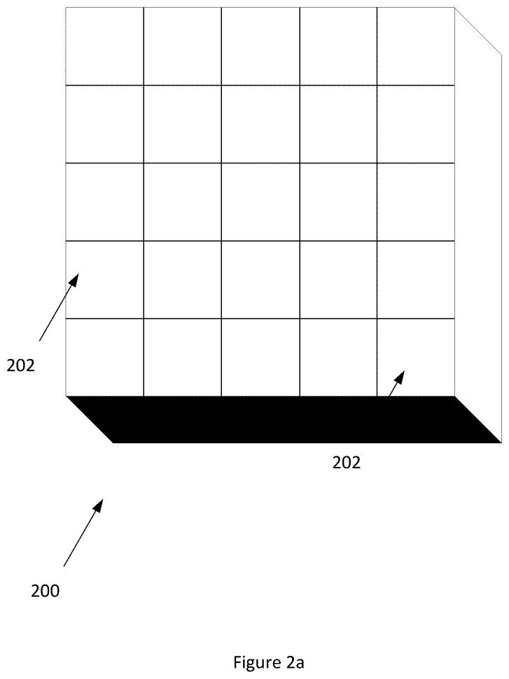

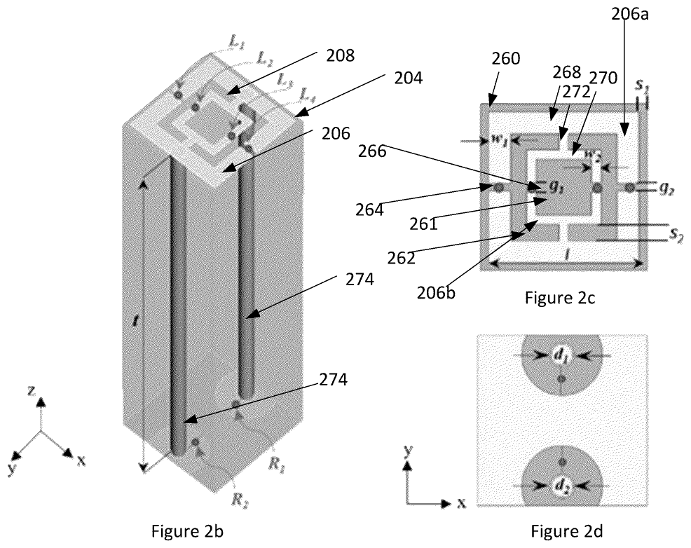

In the current embodiment, the at least one metasurface 42 may be seen as being analogous to an X-ray film used in X-ray mammography. The metasurface 42 includes a set of electrically-small resonators which may be seen as unitcells that are positioned in an organized array. This is described in more detail below with respect to FIGS. 2a to 2d.

In the current embodiment, the low-frequency radiation source 40 is connected to a signal generator 44. The signal generator 44 receives input from a user and transmits signals to the low-frequency radiation source 44 to generate and direct low-frequency electromagnetic waves towards the body part of interest 38 based on the input. In one embodiment, the low-frequency radiation source 40 is fed a continuous wave (CW) signal by the signal generator 44 at a desired frequency, such as, but not limited to, about 100 to about 400 MHz. However, in some embodiments, the frequency or wavelength of the electromagnetic waves is selected such that the size of the wavelengths is larger than a volume or size of the object of interest. For example, if the disclosure is used as a mammography apparatus, the frequency of the waves may be between about 20 MHz to about 400 MHz so that the wavelength of the waves is larger than the volume of the breast being imaged or impressioned.

In the current embodiment, the at least one metasurface 42 is connected to a processing and visualization station 46. The processing and visualization station 46, which in some embodiments, may be a desktop computer, or other similar CPU) receives and then processes the signals received from the metasurface 42 and generates an impression of the body part 38 based on the signals. In some embodiments, the signals may be processed before they are received by the processing and visualization station 46 which then generates an image or impression based on the received processed signals.

The signals that are transmitted by the at least one metasurface 42 are the low-frequency electromagnetic waves received and/or sensed by the at least one metasurface after they have passed through and/or around the body part of interest 38.

While the patient in FIG. 1b is shown as lying down on the platform 32, in other embodiments, there may be a horizontal platform or surface whereby the patient may be standing up and leaning against the horizontal platform with the body part of interest placed between the low-frequency radiation source 40 and the at least one metasurface 42 in an opening within the horizontal platform or surface. The platform may also be positioned at any angle between vertical and horizontal. In yet other embodiments, the opening may be moved within the platform so that a single system 30 can be used to screen different body parts. In some embodiments, when the opening is moved, the metasurface 42 and the low-frequency radiation source 40 also move with the opening 36.

In operation of the embodiment of FIG. 1b, after the patient 34 has been positioned on the platform 32 with their body part 38 placed within the opening 36, the low-frequency radiation source 40 transmits low-frequency electromagnetic waves towards the body part 38 and the metasurface 42 where the electromagnetic waves are larger than a volume or size of the object of interest. The low-frequency electromagnetic waves are generated based on signals or inputs from the signal generator 44 which receives instructions or inputs directly from an individual or remotely from an electronic device (not shown) communicating with the signal generator 44. The metasurface 42 senses or receives the low-frequency electromagnetic waves that pass by and/or through the body part 38. In one embodiment, the metasurface 42 receives the transmitted energy (or low-frequency electromagnetic waves) through the body part in a manner similar to an X-ray mammograph, however, without the harmful radiation that is used by an X-ray mammography machine or apparatus. By placing the metasurface in close proximity to the body part 38 and the low-frequency radiation source 40, the unitcells of the metasurface 42 are able to capture the field information carried by the transmitted energy.

After interaction (the low-frequency electromagnetic waves) with the body part, a power map is absorbed by the metasurface. For testing purposes, the power dissipated in each unitcell (which may be seen as a pixel) can be recorded using a spectrum analyzer or a power meter. In order to see the real-time variations of the power map, each unitcell may be connected to a separate power meter.

Use of the low-frequency radiation source 40 to generate the low-frequency waves, allows higher penetration of the low-frequency waves through dense muscle and/or breast tissue (when the body part of interest is a breast). The low-frequency electromagnetic waves received by the metasurface 42 are then transferred to the processing and visualization station 46 that processes the received signals to generate an impression and/or image of the body part 38 for display to a user of the processing and visualization station 46. As can be seen in FIG. 1b, the processing and visualization station 46 includes a display 48. In one embodiment, the processing and visualization station 26 processes the signals using AI, such as in the form of a convolution neural network (CNN) to characterize the body part and any tumors of anomalies within the body part. It is understood that other neural networks are contemplated. The output of the system may be further processed for visualization. The processing and visualization station 46 may also perform post-processing on the impression that is generated.

In one embodiment of the disclosure, the disclosure generates or creates an impression (or an image) of the breast including its constituents, from which one can determine if the breast has an anomaly (benign or cancerous). The system of FIG. 1b may be seen as an alternative to X-ray based mammography. In X-ray mammography, the breast is illuminated with an X-ray burst of energy and an impression on the other side of the x-ray source is collected via an x-ray sensitive film which may expose to harmful electromagnetic waves.

In the disclosure, a burst of low frequency energy is generated and directed towards one side of the breast and a metasurface (an ensemble of small printed-circuit cells) collects the signals that pass through or around the breast to receive impression signals. In some embodiments, the output from the transmitter, which may be seen as a radio frequency energy source, can be varied. In operation, the impression signals that are sensed or collected by the metasurface may be seen as the power incident on each unitcell. The impression signals are then transmitted to a display/computer for visualization or to a computer for processing to generate an image to be displayed. The resolution of the image or impression is directly dependent on the size of the unitcells that make up the metasurface.

Turning to FIG. 1c, a schematic diagram of yet another embodiment of a system for imaging using low-frequency electromagnetic waves is shown. The current embodiment may be seen as a system for imaging a pipe using low-frequency electromagnetic waves. In some embodiments, the images generated by the system may be used to detect anomalies, such as fractures or cracks in the pipe.

In the system 50 of FIG. 1c, the system 50 includes a transmitter 52 that transmits low frequency electromagnetic waves 54 towards an object of interest, such as pipe 56, which are then sensed or received by a metasurface 58 that is located on a side opposite the transmitter 52 with respect to the pipe 56. The metasurface 58 includes a set of unitcells 59 that receive the signals that pass through and/or around the pipe 56. After receiving the impression signals or electromagnetic waves, the metasurface transmits these readings or signals to a CPU 60 that processes the signals to generate an image or impression of the pipe 56. In the current embodiment, the pipe 56 includes a crack 62 that will be shown in the generated image or impression. The system 50 may further include a signal generator 64 that provides a signal or waveform to the transmitter 52 for the transmission of the electromagnetic waves 54.

Turning to FIG. 2a, a perspective view of a metasurface is shown. As can be seen in FIG. 2a, the metasurface 200 includes a set of unitcells 202 that are positioned in an array of unitcells. In the current embodiment, the metasurface is square-shaped with the unitcells arranged in a 5×5 array. It is understood that if the metasurface is square-shaped, the array of unitcells may be 3×3, 10×10 or any square arrangement based on an application that the metasurface may be used for, however, the metasurface may also be rectangular (i.e. 5×10) or any other shape (i.e. circular where the unit cells are shaped accordingly). In the current embodiment, a surface of each unitcell is square, however, other shaped surfaces of unitcells may be contemplated. In embodiments, the size of the unitcells is smaller than the wavelength of the electromagnetic waves. In some embodiments, for square shaped unitcells, the edges of the unitcells may be between about 2 mm to about 5 mm. It would be understood that a unitcell may have any theoretical size as long as it is smaller than the wavelength of the electromagnetic waves, however, physical limitations affect smaller theoretical implementations of current unitcell design.

The set of unitcells 202 receive the low-frequency energy after the low-frequency electromagnetic waves passed through or around the object of interest. In one specific embodiment, the metasurface 202 includes a 10×10 array of unitcells 202 to provide a metasurface that is comparable in size to a normal female breast, which is around 10 cm in radius. In another embodiment, the metasurface202 may include a predetermined number of unitcells (pixels) to sense a smallest anomaly inside the body part of interest.

Turning to FIGS. 2b to 2d, a perspective view, a top view and a bottom view of a unitcell are shown. Within the metasurface 200, the unitcells 202 are placed in close proximity to each other to produce a tightly-spaced array, whereby the input impedance of each unitcell 202 may be altered or affected by adjacent unitcells within the array. While the unitcell 202 can be designed using different topologies using known technologies, the unitcell in the disclosure includes electric-inductive-capacitive (ELC) resonators that provide high sensitivity to incident electric fields. Components within each unitcell 202 include capacitors and inductors, realized by gaps and conductive ring patterns. In some embodiments, the components are integrated within the unitcell and in other embodiments, the components are surface mounted to the unitcell. One design feature of the metasurface of the disclosure is that the unitcells are low loss to maximize or increase energy absorption by the metasurface 200 at its terminals.

In operation, the unitcells 202 of the metasurface 200 operate as ES antennas. When a breast is the body part being examined, impressioned or imaged, the variation of the permittivity and the conductivity between healthy and cancerous breast tissue can affect the electromagnetic energy (or waves) propagating through the breast and more importantly, the energy that scatters or waves that scatter off the breast tissue that are received by the metasurface whereby the energy received by the metasurface is affected by the presence of a tumor within the breast. The received electromagnetic energy or waves are then processed (such as by the processing station 22) to form an image (impression) that can be processed or reviewed to reveal or determine the presence or absence of an anomaly in the breast.

Furthermore, when an incident electromagnetic field impinges upon the tissue of the body part, the electromagnetic field excites all molecules leading to a secondary scattering from these molecules. This secondary scattering is dependent on the permittivity (related to the polarization of the molecules) and permeability (related to the magnetization of the molecules). Thus each molecule or a cluster of molecules gives a reaction to the impinging electromagnetic field depending on the cluster's constitutive permeability and permittivity parameters thereby enabling the resulting impression of the body part to show the presence or lack of presence of at least one anomaly.

As shown in FIGS. 2b to 2d, the unitcell 202, which may be seen as a miniaturized ELC resonator, includes a top surface 204 which receives the electromagnetic waves or energy that are generated as a result of the low-frequency radiation waves that are directed at the body part of interest. The surface 204 includes a reactive portion 206 for receiving the waves along with a non-reactive portion 208. In one embodiment, the reactive portion 206 includes a set of inductors and resistors for receiving the energy. In one embodiment, the reactive and non-reactive portions are etched onto a printed circuit board.

The non-reactive portion 208 includes an outer ring 260, a central portion 261 and a pair of intermediate portions 262. The outer ring 260 is connected to the pair of intermediate portions 262 via a first pair of interconnects 264 and the pair of intermediate portions 262 are connected to the central portion 261 via a second pair of interconnects 266. The pairs of interconnects 264 and 266 may be seen as gaps separating the reactive portion(s) 206. The reactive portion 206 includes a first section 206a and a second section 206b. Each reactive portion 206a and 206b includes an outer portion 268 and an inner portion 270, the outer portion 268 and the inner portion 270 connected by a reactive interconnect 272.

In the current embodiment, the width of the outer ring 260 is represented as S₁, a width of each of the outer portions 268 is W_{1′} a width of each of the inner portions 270 is W_{2′} a width of each of the first pair of interconnects 264 is G_{2′} a width of each of the second pair of interconnects 266 is G_{1′} and a width of each of the intermediate portions 262 is represented as S₂. The unitcell 202 further includes a pair of vias 274 that are connected to each of the outer portions 268. As shown in FIG. 2d, a diameter of one of the vias is represented as D₁ while a diameter of the other via is represented as D₂.

In order to preserve unitcell symmetry in the x and y directions, the second pair of interconnects 264, or gaps, are positioned between the two outer portions 268 to provide symmetry between the two outer portions. The values of the inductors and resistors for the two different reactive portions 206a and 206b are selected to be the same. In addition, inclusion of the two vias 274 enables the unitcell 202 to maintain a degree of symmetry vis-a-vis the impinging electromagnetic field or the received low-frequency electromagnetic waves. To minimize or reduce loss, in the disclosure, a low-loss Rogers substrate (TMM10i) with a relative permittivity of εᵣ=9.8 and a loss tangent of tan (δ)=0.002 was used. In other words, there is a need to reduce or minimize energy wasted in the metasurface substrate itself and maximize or increase the energy that is absorbed by the sensors (on the back of the metasurface).

In one embodiment, operation of the system may be at a frequency of around 200 MHz which requires a miniaturization of each unitcell 202 such that the metasurface 200 is capable of achieving a high resolution impression. In order to address this, the equivalent capacitance and/or inductance of each unitcell can be increased, thereby decreasing the frequency according to equation (1).

$${f}_{r} = \frac{1}{2 π \sqrt{LC}} , \qquad ( 1 )$$

where fᵣ is the resonance frequency, and C and L are the equivalent capacitance and inductance of an electric-field-coupled (ELC) resonator, respectively. To increase the capacitance, the separation (width of the second pair of interconnects 264) between the metallic parts (or outer portions 268 of reactive portions 206a and 206b) of the unitcell can be decreased, however, this results in an increase in fabrication tolerance and cost. Therefore, in the disclosure, the inductance is increased using lumped inductors placed in the inner and outer rings of the unitcell. In some embodiments, design of a unitcell for a metasurface may be an iterative process to reduce a size of the unitcells while being able to sense waves of specific frequencies,

By manipulating the resonance frequencies of lumped elements, such as, but not limited to, inductors, capacitors, and resistors, for miniaturization of electrically-small resonators or the unitcells, the resonance frequency of each unitcell can be tuned to optimize or obtain desired S-parameters for maximum or improved absorption.

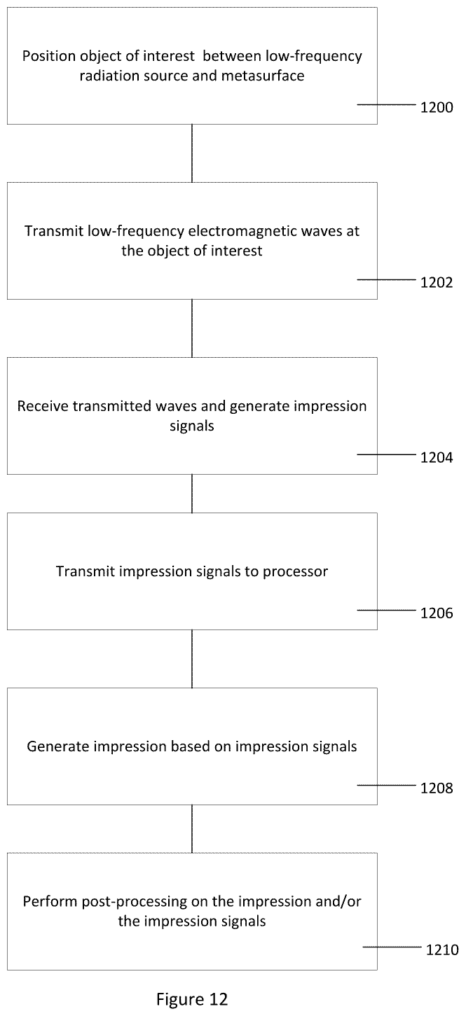

Turning to FIG. 12, a flowchart outlining a method of imaging an object of interest for at least one anomaly using low-frequency electromagnetic waves is shown. Initially, an object of interest that is being screened is placed between a transmitter, such as, but not limited to, a low-frequency radiation source, and a metasurface (1200). Low-frequency electromagnetic waves are then directed towards the object of interest (1202). In one embodiment, the low-frequency electromagnetic waves are transmitted via the low-frequency radiation source via inputs, such a CW wave, from a signal generator. Alternatively, the low-frequency radiation source may receive signals or instructions from an external communication device and generate the low-frequency electromagnetic waves based on the signals of instructions.

The metasurface then receives the transmitted low-frequency electromagnetic waves that have passed through and/or around the object of interest (1204). These received signals may be seen as impression signals or measurements. The impression signals are then transmitted to a processor (1206). In one embodiment, the impression signals are transmitted to the processing station 22.

An impression or image is then generated based on the impression signals (1208). If necessary, or desired, the impression and/or the impression signals may then be further processed (seen as post-processing) to generate or determine other information or data from the impression signals (1210).

In one specific method, when the disclosure is used in mammography, it is assumed that the upper and lower sides of the breast being examined or imaged is pressed and flattened. Thus, in the z-coordinate or z-direction, there is no trouble in adjusting the position of the breast, however with respect to the x- and y-coordinates or directions, it does matter that the left and right breasts are positioned identically with respect to their corresponding coordinate system reference. By doing so, the system is able to eliminate or reduce undesirable background image from the obtained impressions by applying post-processing techniques.

In experiments, an individual unitcell was modeled using electromagnetic field simulation software. As the unitcell is intended to be placed in an infinitely self-repeating structure in both x and y directions, in order to model the periodicity for a unitcell, a perfect or desired magnetic conductor (PMC) boundary condition was placed in the x-direction, and a perfect or desired electric conductor (PEC) boundary condition was placed in the y-direction. The performance of the unitcell was then tested by illuminating the unitcell and recording |S11| (a reflection co-efficient) which represents how much power is reflected or received by the unitcell.

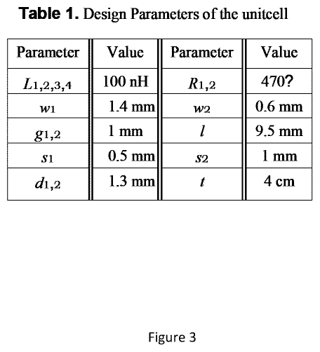

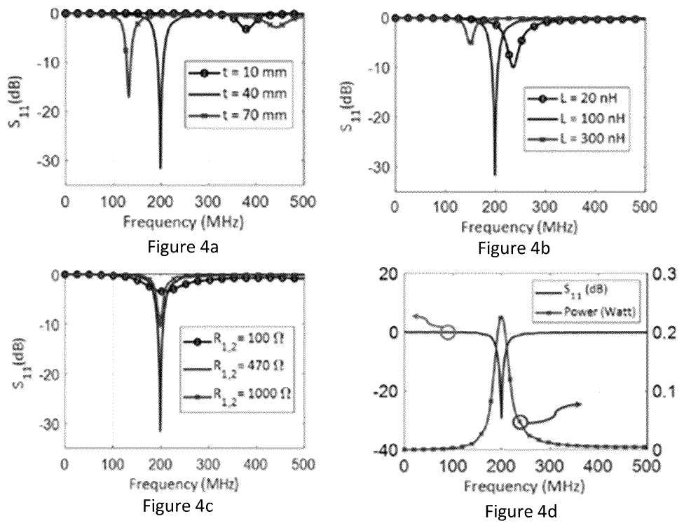

In one embodiment, the parameters of the unitcell may be designed or selected based on two factors, minimum or low |S11| and a maximum or high Q-factor. Examples of calculated parameters of different unitcells are given in the table of FIG. 3. The parameters of FIG. 3 correspond with the symbols in FIGS. 2b to 2d. In one specific embodiment based on the parameters of the table of FIG. 3, a footprint of the unitcell is 10.5 mm×10.5 mm with a thickness of 4.0 cm allowing for increased inductance due to the length of the vias 274. The graphs in FIGS. 4a to 4d provide results from experiments performed using the unitcell with the parameters listed in the table of FIG. 3 where three controlling parameters were investigated. These controlling parameters were the substrate thickness t, the inductors' inductance values L1=L2=L3=L4=L, and the termination resistance values R1=R2.

FIG. 4a shows |S11| on the y-axis and frequency on the x-axis for changing t; FIG. 4b shows |S11| on the y-axis and frequency on the x-axis for changing L; and FIG. 4c shows |S11| on the y-axis and frequency on the x-axis for R1=R2 where the non-variant parameters are set to L=100 nH, R1=R2=470Ω, and t=40 mm. FIG. 4d shows |S11| and power delivered to the terminal resistors of the unitcell, where the incident power is 0.5 Watts.

In FIG. 4a, as a result of the vias adding inductance to the unitcell, the longer the vias are (i.e. higher t), the lower the resonance frequency. FIG. 4b shows that the resonance frequency can be adjusted by varying the inductance of the lumped inductors. FIG. 4c illustrates that the minimum or low |S11| occurs for the value of R1,2=470Ω at the resonance frequency of the unitcell, which indicates perfect impedance matching between the free space and the cell.

FIGS. 4a to 4c also show that impedance matching is affected by the vias' length (t), the inductors' inductance L, and the termination resistors R1=R2, respectively. It was observed that for t=40 mm, L=100 nH, and R1=R2=470 Ω, |S11| has a value of lower than −30 dB, corresponding to a very strong impedance matching at 200 MHZ, and a Q-factor of 16.

FIG. 4d shows the response of the unitcell with the parameters listed in the table of FIG. 3. It can be seen that 0.46 Watts (92%) out of 0.5 Watts incident power is delivered to the termination resistors (at the end of each of the vias) (0.23 Watt in each resistor), and 0.04 Watts (8%) is lost due to dielectric and ohmic loss.

Turning to FIGS. 5a and 5b, top and bottom views of another embodiment of a metasurface are provided. In the current embodiment, the metasurface is a 10×10 array of unitcells, such as the ones described above, and include two termination resistors for each unitcell. As outlined above, the metasurface can include any number of unitcells in any configuration, however, the metasurface of FIGS. 5a and 5b was produced to perform testing of the disclosure. As can be seen in FIG. 5b, each of the unitcells are electrically connected to each of the other unitcells whereby they affect or influence the readings of signals received by adjacent unitcells.

As taught above, each unitcell may be seen as representing a pixel, the value of which is assigned by measuring the power dissipation at the corresponding termination resistors. Owing to the symmetry in the unitcell such as discussed above with respect to FIGS. 2b to 2d, the received power is equally distributed between the resistors. It is understood that non-symmetrical unitcells are also contemplated. By measuring the dissipated power in one of the two termination resistors for each unitcell, a two-dimensional power map (representing the received energy) can be generated, such as by the processing station 26. This two-dimensional power map may be seen as an impression-image of the body part of interest. In other words, the position (xₙ,yₙ) of each unitcell and its termination power dissipation is used for the impression-image construction, which reflects the power absorbed by the each of the unitcells (pixels).

In experiments using the 10×10 array metasurface, this embodiment was leveraged to generate or record a 30×30 impression which enables a larger impression to be generated compared with the number of unitcells used. Considering that the length of the unitcell is Δx, in addition to the reference position, impressions were recorded by shifting the whole metasurface with the value of Δx/3 and 2Δx/3 in both x and y directions. By doing so, nine different combinations of metasurface positions provided nine pixels for each unitcell. Thus, using the whole array, with the same metasurface, an impression with 900 pixels, rather than 100 pixels, was created. An example of the sub-division of unitcells is schematically shown in FIG. 5c.

In the near field, the resolution can dramatically exceed the Abbe diffraction limit. Therefore, if the low-frequency radiation source is placed very close to the body part of interest, such as a breast, the electromagnetic field generated by the low-frequency radiation source that impinges upon the breast contains all polarizations. Therefore, under such excitation, the interaction between the impinging electromagnetic fields and the breast provides a scattered or secondary field that has higher information content.

By recalling the spring and mass model of a molecule interacting with electromagnetic waves, due to the formation of dipole moments inside the breast (or breast model for experiment purposes), molecular polarization takes place. Depending on the polarization of the incident field, molecules inside the breast will be affected differently. Thus, by having different polarizations generated by the low-frequency radiation source, the molecules of healthy and cancerous tissues are excited, thus generating independent information leading to unique impressions that can be generated by the metasurface. As discussed above, an electrically small dipole with a length of 2/10 and a diameter of 2/1000, placed very close to the breast, can be used as the radiation source (and was for some experiments).

In experiments using numerical simulations, various types of breast models were considered in order to test and cover human female breast diversity. Typically, the human breast is mainly composed of fibro-glandular and fat tissues. The density of the breast model can be categorized based on the density of the fibro-glandular tissues. FIG. 5d shows the breast model used by the electromagnetic field simulation software, the breast model including skin, fat tissue, fibro-glandular tissue, and a tumor. Four different categories of the breast model were considered: (1) extremely dense breast (more than 75% fibro-glandular tissue), (2) heterogeneously dense (50% to 75% fibro-glandular tissue), (3) fibro-glandular scattered areas (25% to 50% fibro-glandular tissue), and (4) entirely fatty (less than 25% fibro-glandular tissue).

For the purpose of testing and validating the disclosure, the breast is considered to be a hemisphere with a radius of 50 mm, covered with a layer of skin with a thickness of 2 mm with an internal composition of fibro-glandular and fat tissues. At the operating frequency of 200 MHz, the relative permittivity and electrical conductivity for the fibro-glandular tissue are close to 64 and 0.8 S/m, respectively, whereas for fat tissue, these measurements are close to 5.6 and 0.03 S/m. The center Ct of the spherical tumor, modeled as a perfect conductor (PEC), was placed at (xₜ, yₜ, zₜ) with reference to the origin of the cartesian coordinate system shown in FIG. 5b. This breast model was selected as it provides contrast between the tumor and healthy tissues.

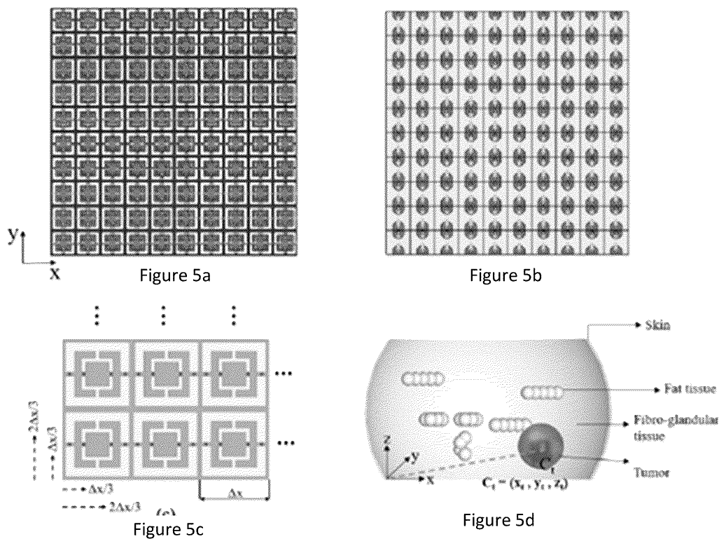

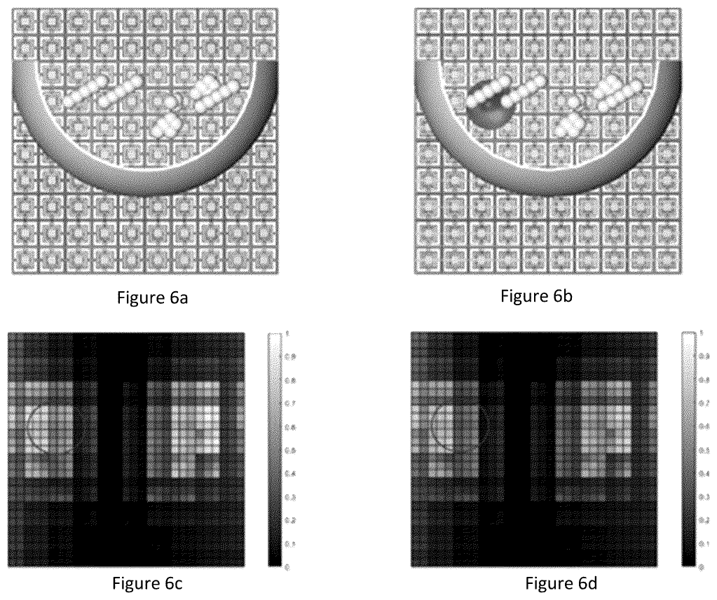

FIGS. 6a and 6b provide simulation models of a healthy breast (FIG. 6a) and a breast with a 10 mm tumor placed in an upper-left corner (FIG. 6b). The table of FIG. 6e lists the different locations of tumors that were used to test the disclosure. FIGS. 6c and 6d are impressions of FIGS. 6a and 6b that were generated by the disclosure. Therefore, FIGS. 6a to 6d show results from a tumor in the Cₜ₁ position of the breast model. As shown in FIGS. 6a to 6b, it can be seen that the differences between the two impressions seem to be negligible to the naked eye. To enhance the resolution, and consequently the potential to increase the differentiation in the impression for the breast with and without the tumor, each impression of a tumorous breast is subtracted from the impression of the same breast but without the tumor such as shown in FIGS. 6c and 6d. In other words, in this embodiment, images of both left and right breasts are obtained and then one image is subtracted from the other image to obtain an impression highlighting a tumor (if present).

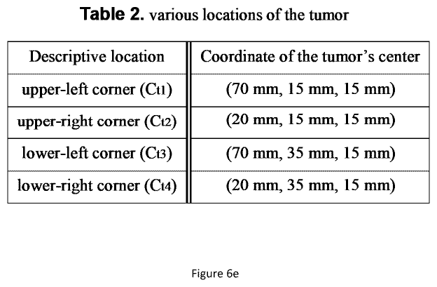

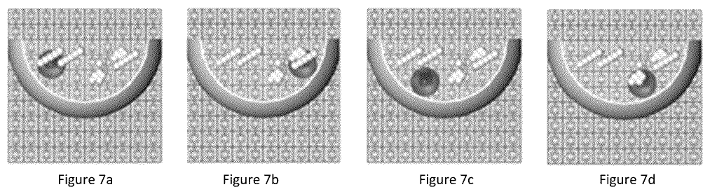

Further experiments were performed with a tumor sample added or placed with different dimensions in various locations. At first, a spherical tumor sample made of PEC with a radius of 10 mm was placed at four different locations: the upper-left corner (Ct₁), the upper-right corner (Ct₂), the lower-left corner (Ct₃), and the lower-right corner (Ct₄) of the breast (as shown in the simulation models of FIGS. 7a to 7d). These locations were selected to show the quality of the impressions and their ability to detect the locations of the tumors and to better observe subtle dissimilitudes between different impressions.

The effectiveness of the subtraction technique, although other calculation techniques are contemplated, is based on the assumption that the left and right breasts are identical for the vast majority of patients. Statistically, the magnitude of relative breast volume (BV) asymmetry between the two breasts has a median of 2.71%, and the magnitude of relative dense volume (DV) asymmetry has a median of 3.28%.

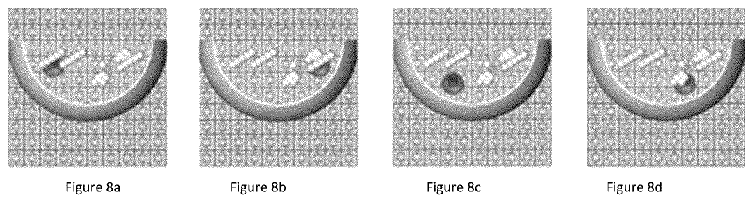

The results of the simulations were analyzed for four different locations of a tumor with a radius of 7.5 mm. The tumor was placed in one of the four positions Cₜ₁, Cₜ₂, Cₜ₃, and Cₜ₄ as depicted in the simulation models of FIGS. 8a to 8d. FIGS. 8e to 8h show the impressions of the above-mentioned tumorous breast models after performing the subtraction technique on the impression of the healthy breast. It was observed that the most pronounced difference was close to the locations of the tumors.

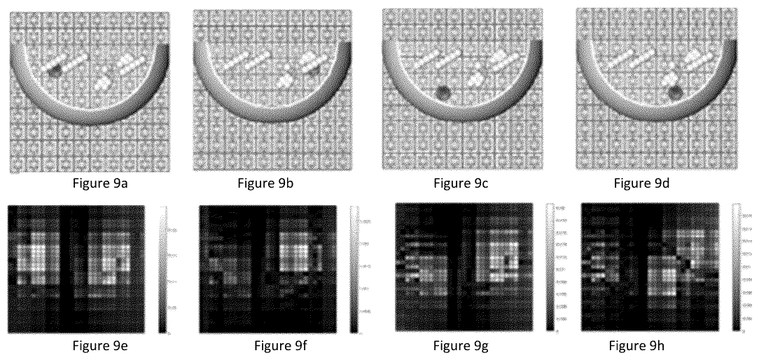

Simulations using the apparatus of the disclosure were also performed for four different locations of a tumor with a radius of 5 mm. The tumor was placed in the same positions (see simulation models of FIGS. 9a to 9d). FIGS. 9e to 9h show the impressions of the corresponding tumorous breast models after applying the subtraction method.

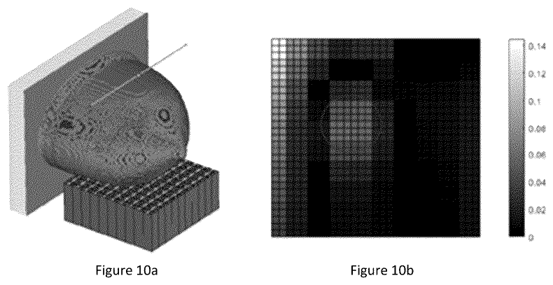

In a further experiment to test the disclosure, a case study was performed for a realistic numerical phantom model. The model that was used was an ACR Class 2-Scattered Fibroglandular breast phantom, the constituents of which have been translated from MRI sagittal slice into electromagnetic field simulation software. The model included different fibroglandular voxel configurations, each of which represented specific electromagnetic characteristics. The operating frequency of the numerical phantom was valid from 0 to 20 GHz theoretically. As shown in FIGS. 10a and 10b, using the method and system of the disclosure, regardless of the breast model under investigation, a desired contrast in the obtained impression (more than DV asymmetry) was obtained. As is shown in FIG. 10b, the contrast that was observed was around 14%, which is above the minimum DV asymmetry of 3.28%.

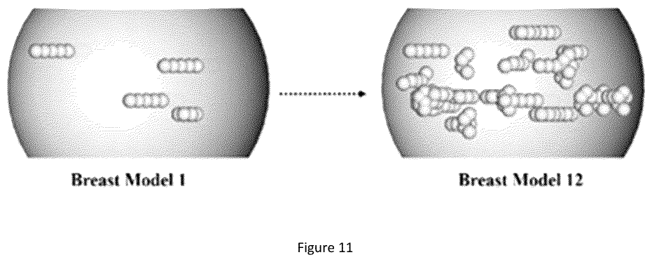

As discussed above, as the disclosure is directed at detecting location and size of possible tumors (and not their shapes or numbers), simulations with spherical tumors and with different radii were performed. The 12 breast models used in our dataset were different in their fat constituents from an extremely dense breast (Breast Model 1) to an entirely fatty (Breast Model 12). Fat tissues were added into the model as experiments were performed between Breast Model 1 and Breast Model 12. In order to make it. FIG. 11 provides a schematic diagram of Breast Model 1 and Breast Model 12.

In other embodiments, the disclosure may be used to generate impressions for any configuration of anomalies with any arbitrary shapes. Furthermore, it is valid for any breast shape with various fibroglandular and fat tissues, as long as the minimum contrast (DV asymmetry) between the anomalies and healthy constituents are provided.

While various embodiments have been described above, it should be understood that they have been presented only as illustrations and examples of the present disclosure, and not by way of limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed:

1. A system for imaging an object of interest comprising:
a low-frequency radiation source for transmitting a set of low-frequency electromagnetic waves towards the object of interest wherein a size of each of the set of low-frequency electromagnetic waves are larger than a volume of the object of interest;
a metasurface for receiving the set of low-frequency electromagnetic waves after they have passed through or around the object of interest to generate impression signals, the metasurface including a set of unitcells; and
a processor for generating at least one impression of the object of interest based on the impression signals.

2. The system of claim 1 further comprising:
a signal generator for providing an input to the low-frequency radiation source.

3. The system of claim 2 wherein the input is a continuous wave signal.

4. The system of claim 2 wherein the input is instructions to generate the set of low-frequency electromagnetic waves.

5. The system of claim 1 further comprising a display for displaying the at least one impression of the object of interest.

6. The system of claim 1 wherein each of the set of unitcells is smaller than the size of each of the low-frequency electromagnetic waves.

7. The system of claim 1 wherein the set of unitcells are arranged in an array.

8. The system of claim 7 wherein each of the set of unitcells comprises:
a reactive portion; and
a non-reactive portion.

9. The system of claim 8 wherein the reactive portion comprises:
an outer portion; and
an inner portion.

10. The system of claim 9 wherein the reactive portion further comprises:
a set of vias connected to the outer portion.

11. The system of claim 1 wherein each of the set of unitcells comprises:
surface mounted components.

12. A method of imaging an object of interest comprising:
transmitting low-frequency electromagnetic waves at an object of interest wherein a size of each of the set of low-frequency electromagnetic waves are larger than a volume of the object of interest;
capturing the low-frequency electromagnetic waves that pass through or around the object of interest with a metasurface including a set of unitcells;
generating impression signals based on the captured low-frequency electromagnetic waves; and
generating an impression based on the impression signals.

13. The method of claim 12 wherein each of the set of unitcells is smaller than the size of each of the low-frequency electromagnetic waves.

14. The method of claim 12 wherein generating impression signals comprises:
processing the captured low-frequency electromagnetic waves.

15. The method of claim 12 wherein transmitting low-frequency electromagnetic waves comprises:
receiving an input at a low-frequency radiation source;
generating the low-frequency electromagnetic waves based on the input; and
transmitting the low-frequency electromagnetic waves at the object of interest.
